(19) United States
(12) Patent Application Publication
(10) Pub. No.: US 2025/0237659 A1
(43) Pub. Date: Jul. 24, 2025

(54) SYSTEMS AND METHODS FOR REDUCING SCATTERING AND IMPROVING OPTICAL POWER DENSITY IN CELLULAR MATRICES

(71) Applicant: The Charles Stark Draper Laboratory, Inc., Cambridge, MA (US)

(72) Inventors: Joseph Hollmann, Cambridge, MA (US); David Sutherland, Cambridge, MA (US)

(21) Appl. No.: 19/035,311

(22) Filed: Jan. 23, 2025

Related U.S. Application Data
(60) Provisional application No. 63/623,957, filed on Jan. 23, 2024.

(51) Int. Cl. G01N 33/58; G01N 21/64

(52) U.S. Cl. CPC G01N 33/582; G01N 21/6428; G01N 21/6452; G01N 2021/6471

(57) ABSTRACT

A system for reducing scattering and improving power density in cellular matrices, may include a light source configured to provide source light at a first wavelength to a cellular matrix, a first filter configured to receive the source light from the light source and provide a first light, a plurality of fluorophores configured to receive at least a portion of the source light and first light and generate a second light at a second wavelength, at least one second filter configured to receive at least a portion of the first light and second light and filter the first light and a detector configured to receive the second light from the at least one second filter to perform optogenetics on the cellular matrices.

### CROSS-REFERENCE TO RELATED APPLICATIONS

This application is a continuation of U.S. Provisional Application No. 63/623,957 filed Jan. 23, 2024, the disclosures of which are hereby incorporated in their entirety by reference herein.

### TECHNICAL FIELD

Aspects of the disclosure generally relate to systems and methods for configuring light in a medium. In particular, the systems and methods disclosed herein relate to enhancing optical power density in a cellular matrix.

### BACKGROUND

A cellular matrix of a medical device can react to light. Light can be altered within the matrix such that it is difficult to detect the light.

### SUMMARY

A system for reducing scattering and improving power density in cellular matrices, may include a light source configured to provide source light at a first wavelength to a cellular matrix, a first filter configured to receive the source light from the light source and provide a first light, a plurality of fluorophores configured to receive at least a portion of the source light and first light and generate a second light at a second wavelength, at least one second filter configured to receive at least a portion of the first light and second light and filter the first light and a detector configured to receive the second light from the at least one second filter to perform optogenetics on the cellular matrices.

In one embodiment, the first filter is a polarization filter configured to provide the first light at a first polarization.

In another embodiment, the at least one second filter includes two filters, wherein one of the filters is a polarization filter.

In one example, the other of the two filter is an absorptive filter to eliminate excitation light due to scattering events.

In another example, the system includes a plurality of scatterers arranged at the cellular matrix configured to return a portion of the light to increase an optical power density of the cellular matrix.

In one embodiment, the system includes a frame arranged an outer edge of the cellular matrix to direct light in a direction opposite that of the source light.

A system of cellular matrices for reducing scattering and improving power density in the cellular matrices may include a plurality of cellular matrices, a light source configured to emit light at the cellular matrices in a first direction along a first path, and a frame arranged around the cellular matrices and configured to direct the light in a second direction along the first path to maintain a threshold power density of the light within the cellular matrices.

In one example, the second direction is opposite the first direction.

In another example, the frame is coupled to the outer edge of the cellular matrix.

In one embodiment, the system includes a first filter configured to receive the light from the light source and provide a first light at a first wavelength.

In another embodiment, the system includes a plurality of fluorophores configured to receive at least a portion of the source light and first light and generate a second light at a second wavelength.

In one example, the system includes at least one second filter configured to receive at least a portion of the first light and second light and filter the first light.

In another example, the system includes a detector configured to receive the second light from the at least one second filter to perform optogenetics on the cellular matrices.

In one embodiment, the system includes a plurality of scatterers arranged at the cellular matrix configured to return a portion of the light to increase an optical power density of the cellular matrix.

In another embodiment the first filter is a polarization filter configured to provide the first light at a first polarization and wherein the at least one second filter includes two filters, wherein one of the filters is a polarization filter.

In one example, the other of the two filter is an absorptive filter to eliminate excitation light due to scattering events.

A system of cellular matrices for reducing scattering and improving power density in the cellular matrices may include a cellular matrix, a light source configured to emit light at the cellular matrices, and a plurality of scatterers disposed within a layer of the cellular matrix and configured to return a portion of the emitted light to the cellular matrix to increase an optical power density of the cellular matrix.

In one example, the system includes a frame arranged an outer edge of the cellular matrix to direct light in a direction opposite that of the source light.

In another example, the frame is coupled to the outer edge of the cellular matrix.

### BRIEF DESCRIPTION OF THE DRAWINGS

The foregoing and other objects, aspects, features, and advantages of the disclosure will become more apparent and better understood by referring to the following description taken in conjunction with the accompanying drawings. The accompanying drawings are not intended to be drawn to scale. Like reference numbers and designations in the various drawings indicate like elements. For purposes of clarity, not every component may be labeled in every drawing. In the drawings:

FIG. 1 illustrates a block diagram of a system for reducing scattering and improving optical power density in cellular matrices;

FIG. 2 illustrates a view of a wavelength filtering scheme for reducing scattering and improving optical power density in cellular matrices;

FIGS. 3A and 3B illustrates a geometry of a cell matrix for reducing scattering and improving optical power density; and

FIG. 4 illustrates a diagram of a scatterer for improving optical power density.

### DETAILED DESCRIPTION

Reference will now be made to the embodiments illustrated in the drawings, and specific language will be used here to describe the same. It will nevertheless be understood that no limitation of the scope of the disclosure is thereby intended. Alterations and further modifications of the features illustrated here, and additional applications of the principles as illustrated here, which would occur to a person skilled in the relevant art and having possession of this disclosure, are to be considered within the scope of the disclosure.

Described herein are systems and methods for reducing scattering and improving optical power density in cellular matrices. Light can scatter within a cellular matrix, causing noise and reducing optical power. The noise and reduction in optical power can pose challenges with a detector configured to receive light of the cellular matrix. Furthermore, size constraints of a cellular matrix can pose difficulties with maintaining sufficient optical power and filtering out undesirable wavelengths of light.

Systems including medical devices configured to have optical detectors for performing optogenetics can be constrained by size requirements. Performing optogenetics can impose limitations of power, sizing, and wavelengths of light and associated device components. Therefore, these systems cannot rely on conventional methods of optical filtering, due to the size and power requirements. Light reaching a detector of an optogenetic system can be off-axis due to scattering and the compressed geometry of the system.

In some cases, light provided to a cellular matrix can be filtered. Examples of conventional filters for optical wavelengths include absorptive and interference filtering. Absorptive filters absorb undesired light and allow other wavelengths to pass. For example, they can reduce unwanted wavelengths of light by ˜2 orders of magnitude. For example, interference filters can reduce unwanted wavelengths of light by ˜8 or more orders of magnitude.

In luminescence imaging/measurements, a fluorophore can be excited with some wavelength and the fluorophore emits a red-shifted light (for example, excitation with a 555 nm light may cause a fluorophore to emit a light of 590 nm). Typically, this fluorescence is orders of magnitude weaker than the excitation signal. It can be desirable in optogenetics to filter the excitation signal's wavelength (in this example, 555 nm) to isolate the fluorescence signal using one of the filters mentioned above. In some cases, these filters have a shifting passband wavelength, dependent on an angle of the incident light. In some cases, this can cause the light to pass through unattenuated. To mitigate this, other conventional systems collimate the light prior to reaching the filter to ensure a desirable angle of the incident light for filtering. In some cases, optical designs ensure light is collimated when it hits the filter through angular filtering of the light. This angular filtering consequentially removes power through a combination of beam stops and distance, weakening the excitation signal and thereby weakening the resulting fluorescence signal.

Furthermore, due to the high absorption of blue light in biological tissue and the presence of significant scattering, transmitting uniform irradiance across thin, high aspect ratio, and dense cellular matrices can be difficult using conventional low-power methods, when constrained to an in-plane orientation of micro-scale optical components. Likewise, when irradiance is used to excite fluorescence species in these high aspect ratio environments, scattering of excitation and background light sources results in high background noise levels that are similarly challenging to filter from fluorescent signals in the constrained environment. Additionally, light can scatter in all directions prior to being reflected back to the detector. For example, a photon can scatter 1000 times before it travels from the light source to the far wall of the cell/hydrogel matrix. Every time light scatters in a conventional system, it can be sent in a different direction. In some cases, the direction of scattering can be given by Mie Scattering. In some cases, after 10 scattering events, the light is likely to be traveling in a random direction (compared to the original unscattered direction). As a result of the above problems, the optical power near the far boundaries of the cell/hydrogel matrix will be much lower than the power near the light source in conventional systems. To account for these and other technical problems, it is desirable to remove the light that was used to excite the fluorescence when it hits the filter at an angle, preserve scattered photons, and ensure a threshold power density across the cell matrix. To do this, the systems and methods described herein can include wavelength filtering which reduces noise and other undesired wavelengths such as polarization and absorptive filtering. This wavelength filtering can be combined with scatterers and a retroreflective frame to remove unwanted signals over a wide range of incident angles and maintain optical power within the cellular matrix.

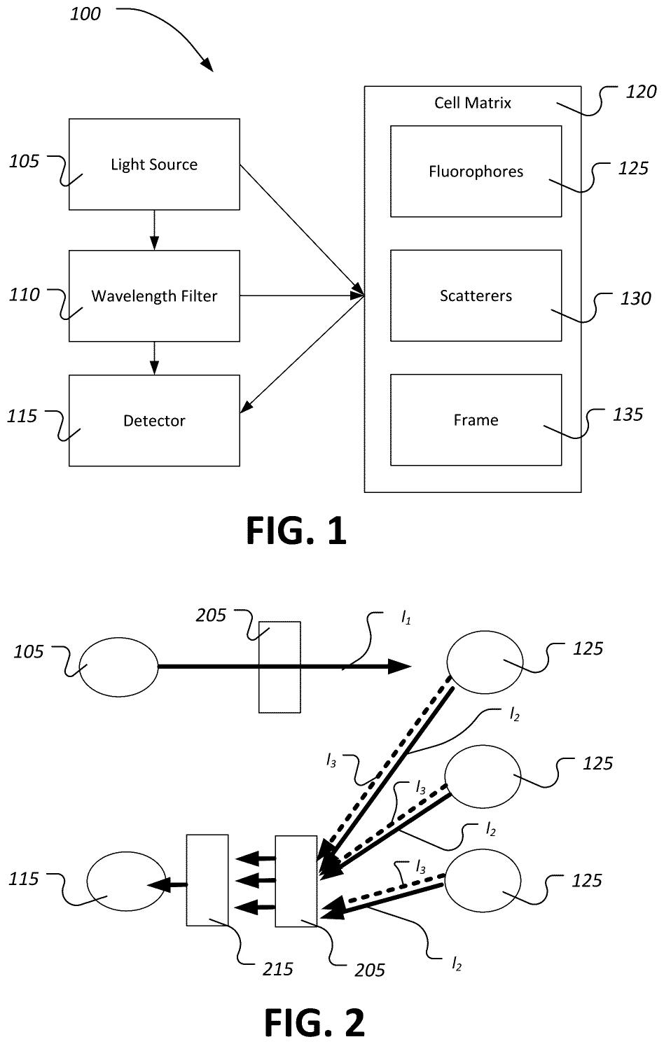

FIG. 1 depicts a block diagram of a system 100 for reducing scattering and improving optical power density in cellular matrices. The system 100 can include a light source 105, wavelength filter 110, a detector 115, or a cell matrix 120, among others. The cell matrix 120 (also herein referred to as the cellular matrix 120, the matrix 120, or the hydrogel matrix 120) can include fluorophores 125, scatterers 130, or a frame 135. The components of the system 100 (e.g., the light source 105, the wavelength filtering 110, the detector 115, or the cell matrix 120) can be coupled with one another.

In brief overview of the system 100, the light source 105 can emit light towards the cell matrix 120. The light source 105 may include fluorescence and bioluminescence light sources. These sources may include mercury arc lamps, Xenon lamps, laser lights, light emitting diode lights sources, infrared lights, etc. Other light sources may also be contemplated such as infrared lights, ultraviolet lights, lamps, etc.

In some cases, the wavelength filter 110 can filter light provided by the light source 105. The wavelength filter 110 may isolate specific ranges of wavelengths or colors from the light source 105. Other wavelengths may be blocked. The wavelength filter 110 may include bandpass filters, low-pass filters, high-pass filters, notch filters, neutral density filter, dichroic filter, polarizing filters, to name a few. In this example, the wavelength filter 110 may be a polarization filter.

In some cases, aspects of the wavelength filtering can be disposed in the cell matrix 120. In some cases, the wavelength filter 110 filters, modifies, or adjusts at least a portion of the light prior to the cell matrix 120 receiving the light provided by the light source 105. The cell matrix 120 can receive the light from the light source 105, from the wavelength filter 110 (i.e., filtered by the wavelength filter 110), or a combination thereof.

The cell matrix 120 can include fluorophores 125 configured to absorb light at specific wavelengths and to emit the light at longer wavelengths. That is, upon receiving a first light in a first wavelength, the fluorophores 125 can absorb at least a portion of the first light and emit a second light in a second wavelength. For example, the fluorophores 125 can undergo luminescence such as fluorescence, phosphorescence, candoluminescence, electroluminescence, among others. The fluorophores 125 may have certain excitation wavelengths and emission wavelengths, as well as other properties such as stokes shift, quantum yield, photostability, brightness, solubility, etc. The fluorophores 125 may be one or more types of fluorophores, including organic, fluorescent proteins, quantum dots, nano-infrared, bioluminescent molecules, etc.

In some cases, the fluorophores 125 can be attached to or associated with one or more cells or a subset of cells, such as human biological tissue, bacteria, fungi, non-human biological tissue, or molecules thereof such as specific proteins or enzymes. In this manner, the fluorophores 125 may mark, tag, or otherwise identify a subset of cells (referred to herein as “the cells”) or other biological molecules. In some cases, the fluorophores 125 are intrinsically coupled with the cells, such as excitable epithelial cells. In some cases, the fluorophores 125 are deposited or added to the cells to provide luminescence.

In some cases, the cell matrix 120 can include scatterers 130 to deflect or disperse the light. The scatterers 130 can be Rayleigh scatterers to ensure symmetric scatter patterns where the intensity may increase as the wavelength decreases. Other scatterers may also be contemplated. The scatterers 130 can scatter any light reflected or transmitted through the cell matrix 120, including the first light and second light as described herein with respect to FIG. 2.

The cell matrix 120 can include a frame 135. The frame 135 can include reflectors, such as retroreflectors. In some cases, the frame 135 can surround at least a portion of the cell matrix 120.

The detector 115 can detect light reflected from the cell matrix 120. The detector may be a photon detector configured to convert light into an electric signal. In some cases, the detector 115 can detect light reflected from the scatterers 130, light reflected from the frame 135 (including retroreflectors), or light emitted by the fluorophores 125. In some cases, the fluorophores 125, the scatters 130, or the frame 135 act to preserve a characteristic of the light emitted by the fluorophores 125 or the light source 105. For example, the scatterers 130 can direct light which may otherwise exit the cell matrix 120 back within the cell matrix to preserve an optical signal of the light. For example, the frame 135 can increase an optical power of the light by changing a direction of the light within the cell matrix 120.

FIG. 2 illustrates a diagram view of a wavelength filtering scheme for reducing scattering and improving optical power density in cellular matrices. As depicted in FIG. 2, the light source 105 provides source light to a first filter 205 at a first wavelength. In some cases, the first filter 205 is a linear polarization filter, though other filters may be considered as outlined above. The first filter 205 can polarize the light. For example, the first filter 205 can shift the light from the light source 105 to a first polarization. In some cases, the polarized light can then be received by the fluorophores 125. In some cases, the light from the first filter 205 can be a first light 11, such as an excitation light. The fluorophores 125 can absorb the excitation light and emit a red-shifted signal that is unpolarized. For example, the fluorophores 125 can absorb at least a portion of the first light 11 and emit a second light 12. In some cases, the second light 12 is not polarized. In some cases, the fluorophores 125 can scatter the polarized excitation light.

The light (e.g., the first light 11, the second light 12, or a combination thereof) can pass through a second filter 210. In some cases, the second filter 210 can also be a polarization filter that is set up to reject the polarized excitation light. The filtered second light 12 (e.g., the first and/or second light) can pass through a third filter 215. The third filter 215 can be an absorptive filter to eliminate any excitation light that might have changed polarization through multiple scattering events.

Polarization filters (e.g., the first filter 205 and/or the second filter 210) have a large acceptance angle (greater than 30 degrees) while retaining the ability to reject a given polarization of light (6 orders of magnitude). The absorptive filter (e.g., the third filter 215) may further attenuate any out-of-band light (2 orders of magnitude). In some cases, the cellular matrix or a part thereof can be thin (e.g., 0.5-1.5 mm thickness) that will highly scatter light. For example, a scattering event can occur, on average, every 1 mm of the cellular matrix. In some cases, the light (e.g., the first light and/or second light) travels between 5 mm to 25 mm.

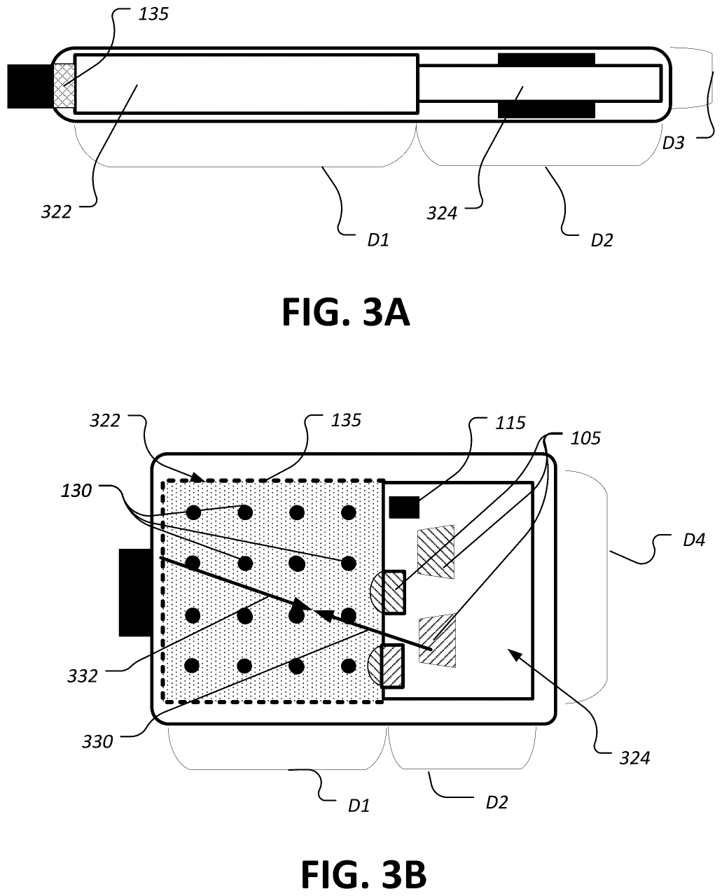

FIGS. 3A and 3 B illustrate a geometry 300 of a cell matrix for reducing scattering and improving optical power density. FIG. 3A illustrates a side view of the cell matrix and FIG. 3B illustrates a top view of the cell matrix 120. The side view can the top view can exhibit the cellular matrix 120.

The cellular matrix 120 may include a first portion 322 containing the cells and hydrogel. This first portion 322 may have a first length, D1. The matrix 120 may include electronics 324 arranged adjacent or on at least a portion of the cells in the first portion 322. The electronics 324 may have a second length, D2. The matrix 120 may have a width, D3. As labeled in FIG. 3B, the matrix 120 may have a width, D4. In some cases, D1 is between 20-30 mm. In some cases, D2 is between 10-20 mm. In some cases, D3 is between 1-4 mm. D4 may be between 10-20 mm, and in this example, may be 15 mm. In one example, D2 is two thirds of D1. For example, D1 may be 15 mm and D2 may be 10 mm. D3 may be 2 mm. D2 and D4 may be the same, where the electronics 324 form a square. The combined length of the electronics 324 and first portion 322 may be between 20-30 mm, or in one example, 25 mm.

The cellular matrix 120 can include the scatters 130. In some cases, the scatters 130 are Rayleigh scatters. In some cases, the scatterers 130 are between 15-25 nm. The scatters 130 may be arranged within or at the cells. The light source 110 may include a plurality of light sources arranged on the electronics 324, or the printed circuit board. The detector 115 may also be arranged on the electronics 324.

The cellular matrix 120 can be coupled with the frame 135. In some cases, the cellular matrix 120 is not enclosed by the frame 135. The frame 135 can include a retroreflector. In some cases, a retroreflector can direct light back the direction it came from. In this manner, this effectively doubles the power density along the edges of the cell/hydrogel matrix. However, it also has the benefit of doubling the optical power where the light has traveled. In some cases, the frame 135 can cause the cellular matrix 120 to maintain an optical power density at or above 4 mW/cm². In some cases, the retroreflector conjugates the phase of the incident optical signal. This means the light can travel back along the exact same path it took to reach the retroreflector. In some cases, the retroreflectors are microbeads. In some cases, the retroreflectors have a diameter between 90-115 μm.

That is, the light source 105 can emit light in a first direction 330 along a first path. The frame can direct the light in a second direction 332 along the first path, and to maintain a threshold power density of the light within the cell matrix. The second direction can be opposite the first direction.

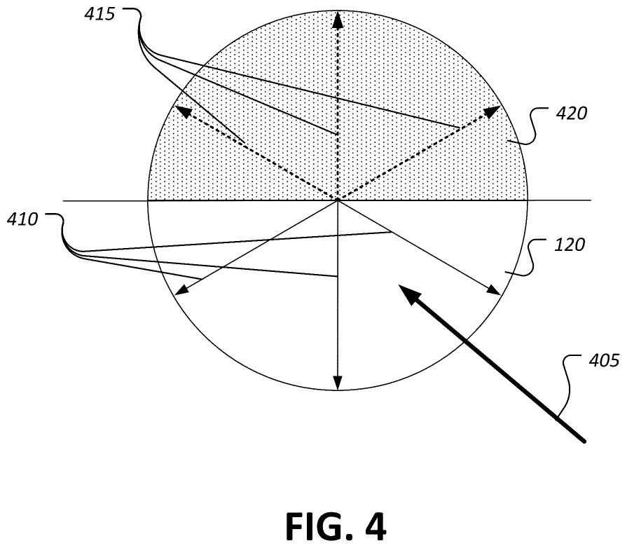

Referring now to FIG. 4, depicted is a diagram 400 of a scatterer 130 for improving optical power density. The scatterer 130 can be coupled to the cellular matrix 120. In some cases, the scatterer 130 is disposed within a layer of the cellular matrix 120. In some cases, the layer has a width and depth between 8-16 mm each. In some cases, the scatterer 120 is chemically or mechanically bound to the layer. In some cases, the scatterer 130 is smaller than a wavelength of light imposed on the cellular matrix. In some cases, the scatterer 130 scatters light equally in all directions.

In some cases, the scatterer 130 can scatter 45-65% of the optical signal leaving the matrix back into the cell/hydrogel matrix. A ray of light 405 can exit the cell/hydrogel matrix and be incident upon the scatterer 130. The scatter 130 can send light equally in all directions, as exhibited by a first set of rays of light 410 and a second set of rays of light 415. The rays of light 415 can be scattered into tissue 420. In some cases, the rays of light 415 are lost due to absorption and represents 45-65% of the light. The rays of light 410 scattered back into the cell/hydrogel matrix can represent ‘scavenged’ optical power that was kept from entering the tissue. This will increase the optical density throughout the cell/hydrogel matrix.

Having now described these technical solutions, it should be appreciated that elements of different embodiments described herein can be combined to form other embodiments not specifically set forth above. Various elements, which are described in the context of a single embodiment, can also be provided separately or in any suitable sub combination. Other embodiments not specifically described herein also within the scope of the following claims.

It is noted that reference to “any” or “or” are not intended to be exclusive; that is to say, any reference to “any” or “or” can encompass all of the elements to which are being referred. Furthermore, various connection and positional relationships (e.g., over, below, adjacent, etc.) are set forth between elements in the above description and in the drawings. These connections or positional relationships, unless specified otherwise, can be direct or indirect, and the described concepts, systems, devices, structures, and techniques are not intended to be limiting in this respect.

Use of ordinal terms such as “first,” second,” “third,” etc. does not by itself connote any priority, precedence, or order of one element over another or the temporal order in which acts are performed, but are used merely as labels to distinguish one element from another similarly named element.

Although the disclosed subject matter has been described and illustrated in the foregoing embodiments, it is understood that the present disclosure has been made only by way of example, and that numerous changes in the details of implementation of the disclosed subject matter may be made without departing from the spirit and scope of the disclosed subject matter.

What is claimed is:

1. A system for reducing scattering and improving power density in cellular matrices, comprising:
a light source configured to provide source light at a first wavelength to a cellular matrix;
a first filter configured to receive the source light from the light source and provide a first light;
a plurality of fluorophores configured to receive at least a portion of the source light and first light and generate a second light at a second wavelength;
at least one second filter configured to receive at least a portion of the first light and second light and filter the first light; and
a detector configured to receive the second light from the at least one second filter to perform optogenetics on the cellular matrices.

2. The system of claim 1, wherein the first filter is a polarization filter configured to provide the first light at a first polarization.

3. The system of claim 1, wherein the at least one second filter includes two filters, wherein one of the filters is a polarization filter.

4. The system of claim 2, wherein the other of the two filter is an absorptive filter to eliminate excitation light due to scattering events.

5. The system of claim 1, further comprising a plurality of scatterers arranged at the cellular matrix configured to return a portion of the light to increase an optical power density of the cellular matrix.

6. The system of claim 1, further comprising a frame arranged an outer edge of the cellular matrix to direct light in a direction opposite that of the source light.

7. A system of cellular matrices for reducing scattering and improving power density in the cellular matrices, comprising:
a plurality of cellular matrices;
a light source configured to emit light at the cellular matrices in a first direction along a first path; and
a frame arranged around the cellular matrices and configured to direct the light in a second direction along the first path to maintain a threshold power density of the light within the cellular matrices.

8. The system of claim 7, wherein the second direction is opposite the first direction.

9. The system of claim 7, wherein the frame is coupled to the outer edge of the cellular matrix.

10. The system of claim 7, further comprising a first filter configured to receive the light from the light source and provide a first light at a first wavelength.

11. The system of claim 10, further comprising a plurality of fluorophores configured to receive at least a portion of the source light and first light and generate a second light at a second wavelength.

12. The system of claim 11, further comprising at least one second filter configured to receive at least a portion of the first light and second light and filter the first light.

13. The system of claim 12, further comprising a detector configured to receive the second light from the at least one second filter to perform optogenetics on the cellular matrices.

14. The system of claim 13, further comprising a plurality of scatterers arranged at the cellular matrix configured to return a portion of the light to increase an optical power density of the cellular matrix.

15. The system of claim 12, wherein the first filter is a polarization filter configured to provide the first light at a first polarization and wherein the at least one second filter includes two filters, wherein one of the filters is a polarization filter.

16. The system of claim 15, wherein the other of the two filter is an absorptive filter to eliminate excitation light due to scattering events.

17. A system of cellular matrices for reducing scattering and improving power density in the cellular matrices, comprising:
a cellular matrix;
a light source configured to emit light at the cellular matrices; and
a plurality of scatterers disposed within a layer of the cellular matrix and configured to return a portion of the emitted light to the cellular matrix to increase an optical power density of the cellular matrix.

18. The system of claim 8, further comprising a frame arranged an outer edge of the cellular matrix to direct light in a direction opposite that of the source light.

19. The system of claim 7, wherein the frame is coupled to the outer edge of the cellular matrix.
